(19) United States
(12) Patent Application Publication
(10) Pub. No.: US 2009/0130752 A1
(43) Pub. Date: May 21, 2009

(54) BIODEGRADABLE POLY(DISULFIDE AMINE)S FOR GENE DELIVERY

(75) Inventors: Sung Wan Kim, Salt Lake City, UT (US); Mei Ou, Salt Lake City, UT (US)

Correspondence Address: ALAN J. HOWARTH, P.O. BOX 1909, SANDY, UT, 84091-1909, US

(73) Assignee: UNIVERSITY OF UTAH RESEARCH FOUNDATION, Salt Lake City, UT (US)

(21) Appl. No.: 12/267,015

(22) Filed: Nov. 7, 2008

Related U.S. Application Data
(60) Provisional application No. 61/002,286, filed on Nov. 7, 2007.

(51) Int. Cl. C12N 15/00; C07C 233/00; C07H 21/00; C12N 15/63; C07H 21/02

(52) U.S. Cl. 435/320.1; 564/153; 564/159; 536/22.1; 536/23.1; 435/455

(57) ABSTRACT

Poly(disulfide amine)s, methods of making, and methods of use are described. Illustrative embodiments of the poly(disulfide amine)s include poly(CBA-DAE), poly(CBA-DAB), and poly(CBA-DAH). These compositions are made by Michael addition between N,N′-cystaminebisacrylamide and N-Boc-protected diamine monomers, followed by N-Boc deprotection. Complexes are formed by mixing the poly(disulfide amine)s with nucleic acid. Delivery of the nucleic acid into cells is carried out by contacting the cells with the nucleic acid/poly(disulfide amine) complexes.

### CROSS-REFERENCE TO RELATED APPLICATIONS

This application claims the benefit of U.S. Provisional Application No. 61/002,286, filed Nov. 7, 2007, which is hereby incorporated by reference in its entirety, except in the event any portion of the provisional application is inconsistent with this application, this application supercedes the provisional application.

### STATEMENT REGARDING FEDERALLY SPONSORED RESEARCH OR DEVELOPMENT

This invention was made with government support under grant no. HL065477 from the National Institutes of Health. The government has certain rights in the invention.

### BACKGROUND OF THE INVENTION

This invention relates to gene delivery. More particularly, this invention relates to nonviral gene delivery carriers.

Gene therapy has broad potential in treatment of human genetic and acquired diseases through the delivery and application of therapeutic gene-based drugs. The use of safe, efficient and controllable gene carriers is a requirement for the success of clinical gene therapy. R. C. Mulligan, The basic science of gene therapy, 260 Science 926-932 (1993); I. M. Verma & N. Somia, Gene therapy-promises, problems and prospects, 389 Nature 239-242 (1997). Although viral vectors are very efficient in gene delivery, their potential safety and immunogenicity concerns raise their risk in clinical applications. C. Baum et al., Mutagenesis and oncogenesis by chromosomal insertion of gene transfer vectors, 17 Hum. Gene Ther. 253-263 (2006). As an alternative to viral vectors, cationic polymers such as poly(L-lysine) (PLL), poly(ethylenimine) (PEI), poly(amidoamine) dendrimers, and cationic liposomes, have been synthesized as gene delivery carriers. The advantages of these cationic polymer carriers include safety, stability, large DNA and RNA loading capacity, and easy and large-scale production. S. Li & L. Huang, Nonviral gene therapy: promises and challenges, 7 Gene Ther. 31-34 (2000); F. Liu et al., Non-immunostimulatory nonviral vectors, 18 Faseb J. 1779-1781 (2004); T. Niidome & L. Huang, Gene therapy progress and prospects: nonviral vectors, 9 Gene Ther. 1647-1652 (2002). The cationic polymers can condense negatively charged DNA into nanosized particles through electrostatic interactions, and the polymer/pDNA polyplexes can enter cells via endocytosis. Y. W. Cho et al., Polycation gene delivery systems: escape from endosomes to cytosol, 55 J. Pharm. Pharmacol. 721-734 (2003); L. De Laporte et al., Design of modular non-viral gene therapy vectors, 27 Biomaterials 947-954 (2006); E. Piskin et al., Gene delivery: intelligent but just at the beginning, 15 J. Biomater. Sci. Polym. Ed. 1182-1202 (2004). As a result, the polymers can protect pDNA from nuclease degradation, and facilitate cellular uptake to induce high gene transfection. O. Boussif et al., A versatile vector for gene and oligonucleotide transfer into cells in culture and in vivo: polyethylenimine, 92 Proc. Nat'l Acad. Sci. USA 7297-7301 (1995); D. W. Pack et al., Design and development of polymers for gene delivery, 4 Nat. Rev. Drug. Discov. 581-593 (2005).

The currently available cationic polymers, however, have significant cytotoxicity concerns, mostly due to their poor biocompatibility and non-degradability under physiological conditions.

Therefore, while prior nonviral gene delivery carriers are known and are generally suitable for their limited purposes, they possess certain inherent deficiencies that detract from their overall utility in gene therapy.

In view of the foregoing, it will be appreciated that providing a biodegradable poly(disulfide amino) gene carrier with high efficiency and low cytotoxicity would be a significant advancement in the art.

### BRIEF SUMMARY OF THE INVENTION

An illustrative embodiment of the present invention comprises a composition represented by the formula

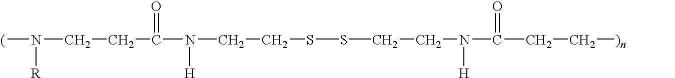

wherein n is about 1 to about 100 and R is (CH₂)ₘNH₂, wherein m is about 1 to about 18. Illustratively, m may be 2, 4, or 6. Typically, n is about 2 to about 50, and, more typically, about 2 to about 20. However, m and n are limited only by the functionality of the composition for use as a nonviral gene delivery carrier.

Another illustrative embodiment of the present invention comprises a method of making a composition represented by the formula

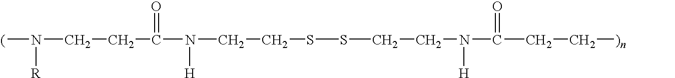

wherein n is about 1 to about 100 and R is (CH₂)ₘNH₂, wherein m is about 1 to about 18, the method comprising:

(a) reacting N,N′-cystaminebisacrylamide with an N-Boc-diaminoalkane to result in

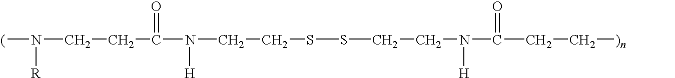

wherein n is about 1 to about 100 and R₁ is BocNH(CH₂)ₘ, wherein m is about 1 to about 18; and

(b) removing the Boc protecting group from R₁ to result in the composition. In one illustrative embodiment of the invention, R is (CH₂)₂NH₂ and the N-Boc-diaminoalkane is N-Boc-1,2-diaminoethane. In another illustrative embodiment of the invention, R is (CH₂)₄NH₂ and the N-Boc-diaminoalkane is N-Boc-diaminobutane. In still another illustrative embodiment of the invention, R is (CH₂)₆NH₂ and the N-Boc-diaminoalkane is N-Boc-diaminohexane.

Still another illustrative embodiment of the present invention comprises a complex comprising a mixture of a selected nucleic acid and a composition represented by the formula

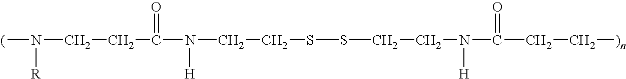

wherein n is about 1 to about 100 and R is (CH₂)ₘNH₂, wherein m is about 1 to about 18. Illustratively, m may be 2, 4, or 6. Typically, n is about 2 to about 50, and, more typically, about 2 to about 20. However, m and n are limited only by the functionality of the composition for use as a nonviral gene delivery carrier.

Yet another illustrative embodiment of the present invention comprises a method for transfecting mammalian cells, the method comprising contacting selected mammalian cells with a complex comprising a mixture of a nucleic acid and a composition represented by the formula

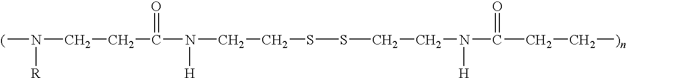

wherein n is about 1 to about 100 and R is (CH₂)ₘNH₂, wherein m is about 1 to about 18. Illustratively, m may be 2, 4, or 6. Typically, n is about 2 to about 50, and, more typically, about 2 to about 20. However, m and n are limited only by the functionality of the composition for use as a nonviral gene delivery carrier.

### BRIEF DESCRIPTION OF THE SEVERAL VIEWS OF THE DRAWINGS

FIG. 1 shows a scheme for synthesis of biodegradable poly(disulfide amine)s.

FIG. 2 shows titration curves obtained by titrating poly(disulfide amine)s in aqueous solutions in 10 mL 0.1 M NaCl from pH 11.0 (initially adjusted with 0.1 M NaOH) to pH 3.0 using 0.01 M HCl: (▪), poly (CBA-DAE); (▴), poly(CBA-DAB); (♦), poly(CBA-DAH). The pH of the solutions was measured after each addition.

FIG. 3 shows average particle sizes of poly(disulfide amine)s/pDNA complexes measured at varying nitrogen/phosphate (N/P) ratios from 1:1 to 80:1, while bPEI (25 kDa)/pDNA complexes were measured at N/P ratios of 10:1 and 20:1. () bPEI; (▴) poly(CBA-DAE); (▾) poly(CBA-DAB); (▪) poly(CBA-DAH).

FIGS. 4A-F shows agarose gel electrophoresis of polyplexes of plasmid DNA with poly(CBA-DAE) (FIGS. 4A and 4D), poly(CBA-DAB) (FIGS. 4B and 4E), and poly(CBA-DAH) (FIGS. 4C and 4F) at different nitrogen/phosphate (N/P) ratios without DTT (FIGS. 4A-4C) and with 5.0 mM DTT (FIGS. 4D-4F) after incubation for 1 hour at 37° C.: lane 1, naked pDNA; lanes 2 and 3, bPEI/pDNA at N/P ratios of 10:1 and 20:1, respectively; lanes 4-11, poly(disulfide amine)s/pDNA at N/P ratios of 1:1, 2:1, 3:1, 5:1, 10:1, 15:1, 20:1, and 40:1, respectively.

FIGS. 5A-D show transfection efficiencies of poly(disulfide amine)s/pDNA polyplexes in a human renal epithelial cell line (293T cells; FIG. 5A); a human cervical cancer cell line (Hela cells; FIG. 5B); a mouse embryonic fibroblast cell line (NIH3T3 cells; FIG. 5C); and a mouse myoblast cell line (C2C12 cells; FIG. 5D) at varying nitrogen/phosphate (N/P) ratios (0.5 μg pDNA/well). Negative controls (C) were untreated cells, and positive controls were cells treated with bPEI 25 kDa at a N/P ratio of 20:1. Results are expressed as the means of triplicate experiments±standard deviations in relative luminescence units (RLU) of luciferase reporter gene expression normalized by total cell protein content in each well.

FIG. 6 shows relative cell viabilities of poly(disulfide amine)s/pDNA polyplexes in NIH3T3 cells at varying nitrogen/phosphate (N/P) ratios compared to a non-treated control group and a bPEI 25 kDa treated group (0.5 μg pDNA/well): (Δ) bPEI, (▾) poly(CBA-DAE), (▪) poly(CBA-DAB), and (∘) poly(CBA-DAH). Cytotoxicity was determined by MTT assay, and data points represent means of triplicate experiments±standard deviations.

### DETAILED DESCRIPTION

Before the present poly(disulfide amine) carriers, complexes, and methods are disclosed and described, it is to be understood that this invention is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

The publications and other reference materials referred to herein to describe the background of the invention and to provide additional detail regarding its practice are hereby incorporated by reference. The references discussed herein are provided solely for their disclosure prior to the filing date of the present application. Nothing herein is to be construed as an admission that the inventors are not entitled to antedate such disclosure by virtue of prior invention.

It must be noted that, as used in this specification and the appended claims, the singular forms “a,” “an,” and “the” include plural referents unless the context clearly dictates otherwise.

Unless defined otherwise, all technical and scientific terms used herein have the same meanings as commonly understood by one of ordinary skill in the art to which this invention belongs.

In describing and claiming the present invention, the following terminology will be used in accordance with the definitions set out below.

As used herein, “comprising,” “including,” “containing,” “characterized by,” and grammatical equivalents thereof are inclusive or open-ended terms that do not exclude additional, unrecited elements or method steps. “Comprising” is to be interpreted as including the more restrictive terms “consisting of” and “consisting essentially of.” As used herein, “consisting of” and grammatical equivalents thereof exclude any element, step, or ingredient not specified in the claim. As used herein, “consisting essentially of” and grammatical equivalents thereof limit the scope of a claim to the specified materials or steps and those that do not materially affect the basic and novel characteristic or characteristics of the claimed invention.

The present invention relates to a series of linear cationic polymers with many of the characteristics of ideal polymeric gene delivery carriers that can mediate high gene transfection with low cytotoxicity. Advantages of these polymers are as follows. (1) Defined and improved polymer structures. Prepared by Michael addition and N-Boc deprotection under acidic condition, these polymers contain disulfide bonds, tertiary amine groups, and pendant primary amine groups in structures, and they do not form uncontrollable branches and crosslinking in synthesis. These structures aim to meet the fundamental design criteria of good gene carriers: reasonable biodegradability, strong DNA condensation ability, efficient gene transfection, and low cytotoxicity. (2) Biodegradability. Poly(disulfide amine)s contain disulfide bonds in the main chain, and are relatively stable in the extracellular oxidizing environment while being rapidly degraded in the intracellular reducing environment. Therefore, genetic materials in polyplexes will be released efficiently in the cytoplasm to allow for efficient gene expression. Meanwhile, cytotoxicity will decrease due to polymer degradation. (3) High nucleic acid binding affinity. Introducing unique primary amine side groups into poly(disulfide amine)s improves water solubility and enhances positive-charge density. This allows plasmid DNA and other genetic materials, such as antisense oligonucleotides, peptide nucleic acids, and siRNA, to be stably condensed into nanosized particles under physiological pH, which will contribute to endocytosis and consequently efficient gene transfection. (4) High buffering capacity. The combination of tertiary and primary amine groups in poly(disulfide amine)s can promote endosomal-lysosomal escape based on the “proton sponge hypothesis”. This characteristic gives poly(disulfide amine)s great potential in gene delivery.

Herein are described illustrative poly(disulfide amine)s that were synthesized via Michael addition and N-Boc deprotection. Polymers were characterized by ¹H NMR, SEC, and acid-base titration. The properties of polymer/pDNA complexes were studied by dynamic light scattering and gel electrophoresis. In vitro transfection as well as in vitro cytotoxicity of polymer/pDNA complexes were evaluated by luciferase assay, BCA protein assay, and MTT assay using 293T cells (human renal epithelial cell line), Hela cells (human cervical cancer cell line), NIH3T3 (mouse embryonic fibroblasts), and C2C12 cells (mouse myoblast cell line).

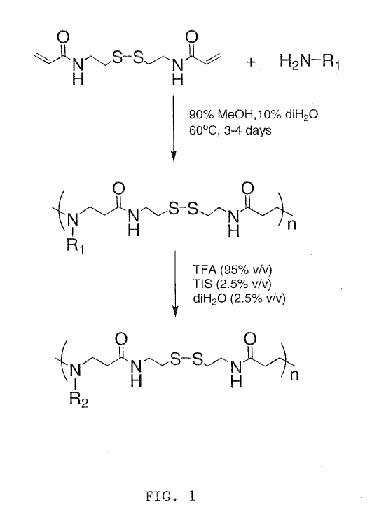

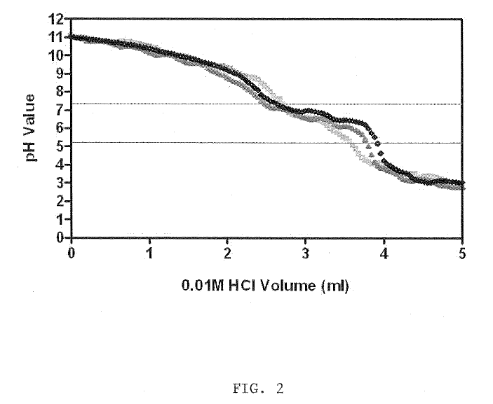

Three illustrative biodegradable polydisulfide amines were synthesized (Examples 1-3) by Michael addition between N,N′-cystaminebisacrylamide (CBA) and three different N-Boc-protected diamine monomers, N-Boc-DAE, N-Boc-DAB, and N-Boc-DAH. After removing N-Boc protection groups, three linear comb-like polymers, poly(CBA-DAE), poly(CBA-DAB) and poly(CBA-DAH), were synthesized with one disulfide bond, one tertiary amine group in the main chain, and one pendant primary amine group in the side chain in each repeating units (FIG. 1). All three polydisulfide amines were purified by dialysis and were then lyophilized to yield solid powders. These polydisulfide amines were readily soluble in water, PBS buffer, HEPES buffer, Tris buffer, dimethyl sulfoxide (DMSO), and methanol, but not in chloroform, diethyl ether, or tetrahydrofuran. The final structures of these polydisulfide amines were confirmed by ¹H NMR (400 MHZ, D₂O; Example 4). The disappearance of signal peaks between δ5 to 7 ppm indicated that the acrylamide end groups no longer existed in the final polymer products. Additionally, the ¹H NMR results confirmed that the polymers had the expected defined structures, and no branches were observed.

The molecular weight of polymers were measured by fast protein liquid chromatography (FPLC) and calibrated by pHPMA standards (Table 1; Example 5). The range of the weight average molecular weight (M_{w}) of these polymers was from 3.34˜4.72 kDa, while the range of the number average molecular weight (Mₙ) was from 2.85˜4.23 kDa. The polydispersity index (PDI=M_{w}/Mₙ), ranging from 1.12˜1.17, indicates that these poly(disulfide amine)s have a narrow molecular weight distribution.

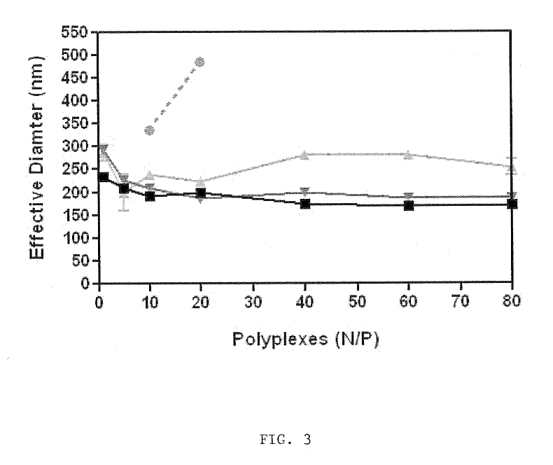

Buffering capacity is an important factor for cationic gene carriers according to the “proton sponge hypothesis.” O. Boussif et al., supra. It helps polymeric carriers to effectively compact and protect DNA after endocytosis, and helps DNA escape from endosomes-lysosomes. T. G. Park, J. H. Jeong & S. W. Kim, Current status of polymeric gene delivery systems, 58 (Adv. Drug Deliv. Rev. 467-486 (2006); N. D. Sonawane et al., Chloride accumulation and swelling in endosomes enhances DNA transfer by polyamine-DNA polyplexes, 278 J. Biol. Chem. 44826-44831 (2003); Z. Zhong, J. Feijen, M. C. Lok, W. E. Hennink, L. V. Christensen, J. W. Yockman, Y. H. Kim & S. W. Kim, Low molecular weight linear polyethylenimine-b-poly(ethylene glycol)-b-polyethylenimine triblock copolymers: synthesis, characterization, and in vitro gene transfer properties, 6 Biomacromolecules 3440-3448 (2005). Buffering capacities of poly(disulfide amine)s, measured by acid-base titration, were expressed as the percentage of amine groups becoming protonated from pH 7.4 to 5.1, mimicking the change from the high pH extracellular environment to the low pH endosomal environment. C. Lin et al., supra; L. V. Christensen et al., supra; A. Akinc et al., supra. The results (Table 1 and FIG. 2) show that all three illustrative poly(disulfide amine)s have excellent buffering capacity, ranging from 52.61% to 61.20% protonation, which is much higher than the previously reported results (24%) of bPEI 25 kDa, C. Lin et al., supra, and 13.5%, L. V. Christensen et al., supra. The high buffering capacities enable poly(disulfide amine)s to facilitate endosomal escape, contributing to an increase in gene transfection efficiency.

There are several attributes of linear poly(disulfide amine)s that make them particularly attractive as polymeric gene carriers: (1) the polymers contain disulfide bonds for fast biodegradation; (2) primary and tertiary amine groups can self-assemble with DNA at physiological pH, facilitating endosomal escape and efficient release of DNA to the nucleus; (3) primary amine groups at each repeating unit provide for high nucleic acid binding affinity and good water solubility; (4) a variety of analogues can potentially be synthesized directly from commercially available monomer materials; and (5) amine concentration can be evaluated for more accurate and efficient gene transfection.

To mediate endocytosis through cell membrane, cationic polymers need to condense DNA into nanosized particles via electrostatic interactions between the positive charged polymers and the negative charged phosphates on DNA backbones. D. W. Pack et al., 4 Nat. Rev. Drug Discov. 581-593 (2005); D. Oupicky et al., Laterally stabilized complexes of DNA with linear reducible polycations: strategy for triggered intracellular activation of DNA delivery vectors, 124 J. Am. Chem. Soc. 8-9 (2002). Dynamic light scattering (DLS) studies (Example 6) showed that three illustrative poly(disulfide amine)s can condense plasmid DNA to small particles with effective diameters less than 300 nm at polymer/pDNA nitrogen/phosphate (N/P) ratios of 1:1 and above. In contrast, the diameters of bPEI/pDNA particles were larger at N/P ratios of 10:1 and 20:1 (336.5 nm and 484.5 nm) under the same measuring condition (FIG. 3).

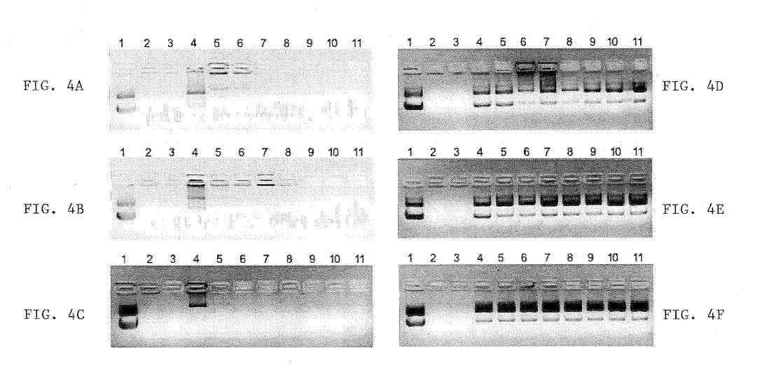

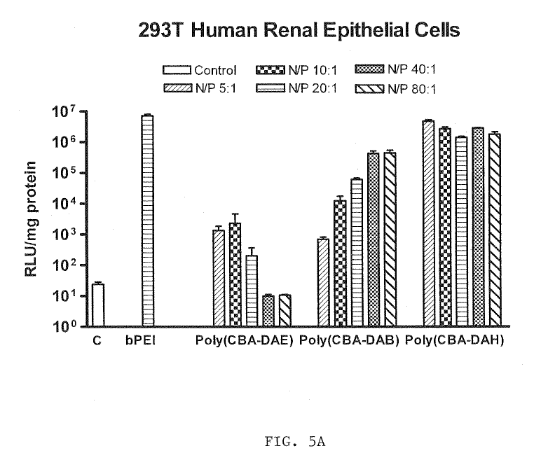

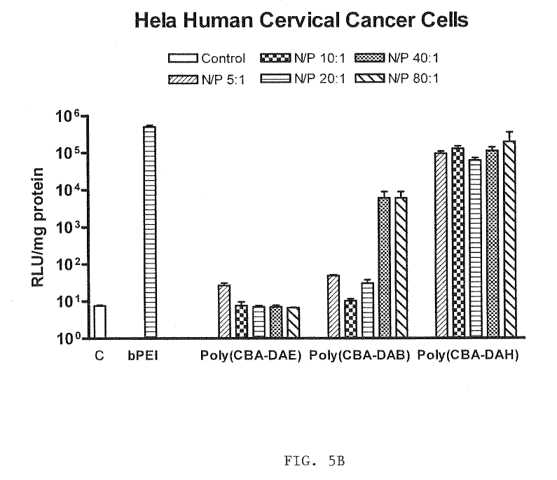

Gel retardation assay (Example 7) further verified that illustrative poly(disulfide amine)s can condense plasmid DNA at low N/P ratios. All three illustrative poly(disulfide amine)s were dissolved in HEPES buffer solution (20 mM HEPES, pH 7.4, 5% glucose). One μg plasmid DNA (pCMV-Luc) per sample with varying amount of polymers were mixed and incubated at desired N/P ratios, followed by performing agarose gel electrophoresis and staining with ethidium bromide (EtBr) (FIGS. 4A-4F). In the absence of DTT (FIGS. 4A-4C), poly(CBA-DAE), poly(CBA-DAB), and poly(CBA-DAH) can completely retard plasmid DNA migration from N/P ratios of 5:1, 3:1, 3:1, respectively. When the polyplexes were incubated with 5.0 mM DTT at 37° C. for 1 hr, mimicking the intracellular reducing environment containing 0.1-10 mM glutathione, as expected, pDNAs were released from all three poly(disulfide amine)s at all N/P ratios, with bands migrating toward to positive electrode in gel electrophoresis (FIGS. 4D-4F). For the non-degradable control polymer bPEI 25 kDa, there was no pDNA released from bPEI/pDNA complexes in the presence of DTT. This gel retardation assay proved that all three illustrative poly(disulfide amine)s can release pDNA efficiently from polyplexes via disulfide bonds cleavage, leading to increased DNA release so as to increase gene expression.

To facilitate efficient gene expression, cationic polymers should not only strongly condense plasmid DNA extracellularly, but also efficiently release DNA from polyplexes intracellularly. Previously, the hydrolysable polymers, such as poly(β-amino amine)s and poly(amido amine)s, were synthesized by one-step Michael addition and only contained tertiary amines, hydroxyl and/or imidazole groups. D. G. Anderson et al., 42 Angew Chem. Int. Ed. Engl. 3153-3158 (2003); C. Lin et al., supra. The tertiary amine groups have limited DNA binding affinity due to steric hindrance, while hydroxyl and imidazole groups contribute little in binding DNA. As a result, relatively high N/P ratios were required to completely condense DNA. For example, to retard DNA migration in agarose gel, weight ratios equal to or higher than 40:1 were needed for poly(β-amino amine)s. D. G. Anderson et al., 11 Mol. Ther. 426-434 (2005). Similarly, weight ratios of 24:1 or higher are required for poly(amido amine)s, such as pAPOL. C. Lin et al., supra. For the poly(disulfide amine)s, on the contrary, the results of gel retardation assay showed that they can form stable complexes with pDNA at N/P ratios as low as 3:1, suggesting that poly(disulfide amine)s with primary amines have stronger nucleic acid binding affinities than those hydrolysable polycations as mentioned above. In addition, some hypotheses indicated that pendant primary amine groups are more nucleophilic than tertiary amine groups, which will facilitate more efficient gene transfection and expression. A. Akinc et al., supra. It is also well known that disulfide bonds can be cleaved rapidly in the presence of intracellular high concentration of glutathione and thioredoxin reductases. This rapid cleavage of disulfide bonds will ensure DNA release from complexes efficiently so as to facilitate nuclear import, gene transcription, and gene expression to occur. C. Lin et al., supra; L. V. Christensen et al., supra; C. Pichon et al., Poly[Lys-(AEDTP)]: a cationic polymer that allows dissociation of pDNA/cationic polymer complexes in a reductive medium and enhances polyfection, 13 Bioconjug. Chem. 76-82 (2002); X. L. Wang et al., A novel environment-sensitive biodegradable polydisulfide with protonatable pendants for nucleic acid delivery, 120 J. Control. Rel. 250-258 (2007). The presently described poly(disulfide amine)s also showed the ability for rapid cleavage in a reducing environment, so they are expected to have good ability for inducing high gene expression. In summary, poly(disulfide amine)s demonstrated strong DNA condensing abilities by forming nanosized particles at low N/P ratios. They also showed rapid DNA releasing abilities by rapid disulfide bonds cleavage in reducing environment.

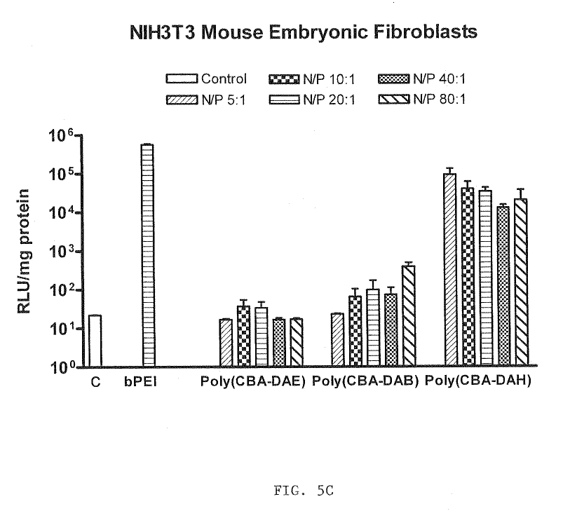

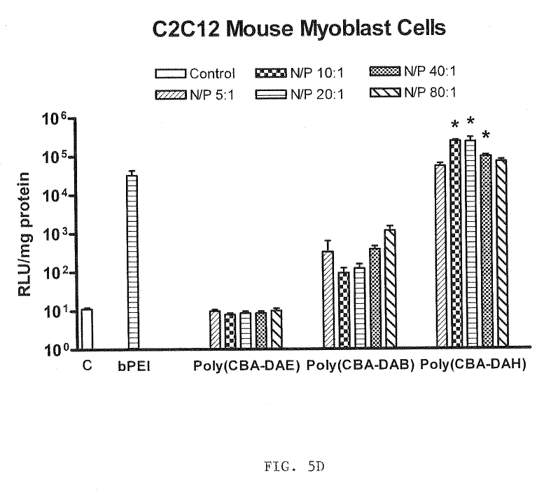

To evaluate in vitro transfection efficiency of biodegradable poly(disulfide amine)s, their complexes with reporter gene pCMV-Luc (0.5 μg/well) expressing luciferase were conducted on four different cell lines, 293T, Hela, NIH3T3, and C2C12, at five N/P ratios ranging from 5:1 to 80:1 in the absence of serum (Example 8). Complexes of bPEI (25 kDa)/pDNA at an N/P ratio of 20:1 were used as a positive control. At this N/P ratio, bPEI showed the highest gene transfection efficiency while maintaining at least 70% cell viability. The transfection efficiency was quantitatively measured as luciferase enzyme activity and normalized as total cell protein concentration by BCA protein assay (FIGS. 5A-D). Among these poly(disulfide amine)s, poly(CBA-DAH) showed the highest level of gene expression in all four cell lines. In 293T, Hela, and NIH3T3 cell lines, poly(CBA-DAH) had comparable luciferase gene expression level to bPEI 25 kDa, at varying N/P ratios from 5:1 to 80:1. Interestingly, poly(CBA-DAH) expressed up to 7-fold higher gene transfection efficiency than bPEI in the C2C12 cell line at all N/P ratios, which was statistically significant. The mouse myoblast C2C12 cell line is generally a cell line that is difficult to transfect with cationic polymers.

For the three exemplary poly(disulfide amine)s, the transfection efficiency sequences are: poly(CBA-DAH)>poly(CBA-DAB)>poly(CBA-DAE). The main difference among the three polymers is their side chain lengths, suggesting that the side chains will influence gene transfection efficiency, D. G. Anderson et al., 11 Mol. Ther. 426-434 (2005). Poly(CBA-DAH) has a longer alkyl chain between the tertiary and the primary amine groups than those of poly(CBA-DAE) and poly(CBA-DAB). It is speculated that poly(CBA-DAH) was more efficient due to its interaction with the lipid bilayer of cell membrane via hydrophobic interactions, as compared to poly(CBA-DAE) and poly(CBA-DAB), since the longer chain introduces more flexibility and hydrophobicity into the polymer. These results suggest that it may be important to optimize side chain structures to achieve high transfection efficiency.

The high gene transfection efficiency of poly(CBA-DAH) is comparable to bPEI 25 kDa, especially in the C2C12 cell line. This can be explained by the following reasons: (1) poly(CBA-DAH) contains tertiary and primary amine groups and flexible side chains, so it has excellent buffering capacity to help plasmid DNA escape from endosomes after endocytosis of the polyplexes based on proton sponge effects; (2) the disulfide bonds in the main chain of poly(CBA-DAH) can be rapidly cleaved by the high endosomal concentration of glutathione and thioredoxin reductases, so that DNA can be efficiently released from polyplexes to increase gene expression.

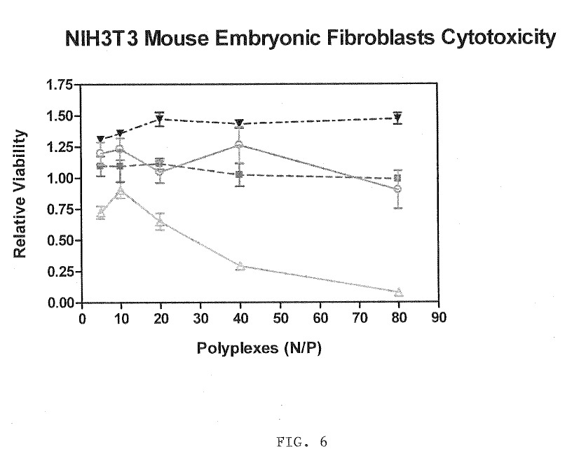

In vitro cytotoxicity of poly(disulfide amine)s was evaluated by a standard MTT assay on NIH3T3 cells (FIG. 6; Example 9). The experiments were performed the same manner as the transfection experiments described above, except that the MTT assay was performed at 24 hrs instead of 48 hrs post-transfection. Poly(disulfide amine)s showed low toxicity compared to bPEI 25 kDa. The overall profile in FIG. 6 showed that bPEI 25 kDa has increasing cytotoxicity with the increasing N/P ratios, while cell viability decreased to 7.7% at N/P ratio of 80:1. In contrast, poly(disulfide amine)s showed no significant toxicity for cells even at N/P ratio of 80:1, retaining 90% or higher cell viability relative to control cells (non-treated NIH3T3 cells). These results are consistent with other three cell lines: 293T, Hela, and C2C12. In conclusion, these poly(disulfide amine)s are far less cytotoxic than bPEI 25 kDa, suggesting that poly(disulfide amine)s are readily degraded into non-toxic small molecules after endocytosis.

In summary, these poly(disulfide amine)s, especially poly(CBA-DAH), have high gene transfection efficiency and low cytotoxicity and great potential for gene delivery in vitro. From the above data, poly(CBA-DAH) exhibits significant high gene transfection in mouse myoblasts (C2C12 cells). These poly(disulfide amine)s are likely to be effective gene carriers in many other primary cells and stem cells. It has been shown that poly(CBA-DAH) has high gene transfection efficiency on SVR cells (mouse pancreatic islet endothelial cells). Beside delivering plasmid DNA, poly(disulfide amine)s can be used as gene carriers to deliver other types of genetic materials into human cells, such as antisense oligonucleotides, therapeutic genes, and small interfering RNA (siRNA). Furthermore, poly(disulfide amine)s can be modified with targeting moieties to specifically delivery genetic materials into certain cell types.

### EXAMPLES

Materials. tert-Butyl N-(2-aminoethyl)carbamate (N-Boc-1,2-diaminoethane, N-Boc-DAE), tert-butyl N-(4-aminobutyl)carbamate (N-Boc-1,4-diaminobutane, N-Boc-DAB), tert-butyl-N-(6-aminohexyl)carbamate (N-Boc-1,6-diaminohexane, N-Boc-DAH), hyperbranched polyethylenimine (bPEI, M_{w}=25 kDa), trifluoroacetic acid (TFA), triisobutylsilane (TIS), dithiothreitol (DTT), ethidium bromide (EtBr), and 3-(4,5-dimethyl-2-thiazolyl)-2,5-diphenyl-2H-tetrazolium bromide (MTT) were from Sigma-Aldrich (St. Louis, Mo.). N,N′-Cystaminebisacrylamide (CBA) was from PolySciences, Inc. (Warrington, Pa.). The plasmid, pCMV-Luc, containing a firefly luciferase reporter gene inserted into a pCI plasmid vector driven by the CMV promoter (Promega, Madison, Wis.), was amplified in E. coli DH5α and isolated with a Maxiprep kit (Invitrogen, Carlsbad, Calif.) according to the manufacturer's instructions. Dulbecco's Modified Eagle's Medium (DMEM), penicillin-streptomycin, fetal bovine serum (FBS), trypsin-like enzyme (TrypLE Express), and Dulbecco's phosphate buffered saline (PBS) were from Invitrogen-Gibco (Carlsbad, Calif.). Luciferase assay system with reporter lysis buffer was from Promega (Madison, Wis.). All materials and solvents were used as received without further purification.

### Example 1

The scheme for synthesis of poly(disulfide amine)s according to the present invention is illustrated in FIG. 1. In this example, the synthesis of poly(CBA-DAE) is described. Briefly, N-Boc-DAE (0.160 g, 1 mmol) and CBA (0.260 g, 1 mmol) were placed in a flask and dissolved in 1 mL MeOH/H₂O (9/1 v/v). Polymerization was conducted in an oil bath at 60° C. in the dark under a nitrogen atmosphere for 4 days. Then, a 10% molar excess of N-Boc-DAE was added to the reaction solution to consume any unreacted acrylamide functional groups, and the reaction was performed at 60° C. for at least an additional 2 hrs. After that, the product was precipitated with 40 mL anhydrous diethyl ether and dried. Subsequently, the acid-labile N-Boc protection group was removed with TFA/TIS/H₂O (95/2.5/2.5 v/v) for 30 min. The crude product was precipitated in 40 mL anhydrous diethyl ether and dried under vacuum. It was further purified by dialysis (MWCO=1000) against MilliQ deionized water overnight, followed by lyophilization to obtain poly(CBA-DAE) as a solid powder.

### Example 2

Poly(CBA-DAB) was synthesized according to the procedure of Example 1, except that polymerization was for three days.

### Example 3

Poly(CBA-DAH) was synthesized according to the procedure of Example 1, except that polymerization was for three days.

### Example 4

The poly(disulfide amine)s prepared according to Examples 1-3 were analyzed by ¹H NMR (400 MHZ, D₂O), and the data were listed as following:

Poly(CBA-DAE) 2.91 (NCH₂CH₂NH₂, 2H), 2.64 (NCH₂CH₂NH₂, 2H), 2.63 (NCH₂CH₂CO, 4H), 2.22 (NCH₂CH₂CO, 4H), 3.34 (CONHCH₂CH₂SS, 4H), 2.62 (CH₂SSCH₂, 4H);

Poly(CBA-DAB) 3.08 (NCH₂CH₂CH₂CH₂NH₂, 2H), 1.58 (NCH₂CH₂CH₂CH₂NH₂, 2H), 1.58 (NCH₂CH₂CH₂CH₂NH₂, 2H), 2.91 (NCH₂CH₂CH₂CH₂NH₂, 2H), 2.82 (NCH₂CH₂CO, 4H), 2.48 (NCH₂CH₂CO, 4H), 3.38 (CONHCH₂CH₂SS, 4H), 2.63 (CH₂SSCH₂, 4H);

Poly(CBA-DAH) 3.15 (NCH₂CH₂CH₂CH₂CH₂CH₂NH₂, 2H), 1.48 (NCH₂CH₂CH₂CH₂CH₂CH₂NH₂, 2H), 1.19 (NCH₂CH₂CH₂CH₂CH₂CH₂NH₂, 2H), 1.19 (NCH₂CH₂CH₂CH₂CH₂CH₂NH₂, 2H), 1.48 (NCH₂CH₂CH₂CH₂CH₂CH₂NH₂, 2H), 2.85 (NCH₂CH₂CH₂CH₂CH₂CH₂NH₂, 2H), 2.81 (NCH₂CH₂CO, 4H), 2.52 (NCH₂CH₂CO, 4H), 3.35 (CONHCH₂CH₂SS, 4H), 2.65 (CH₂SSCH₂, 4H).

### Example 5

The molecular weights and polydispersity of the polymers prepared according to Examples 1-3 were determined by size exclusion chromatography (SEC) on an AKTA FPLC system (Amersham Biosciences, Piscataway, N.J.) equipped with a Superose® 12 column and UV and refractive index detectors. The polydisulfide amines were dissolved in 0.5 mL of Tris buffer (pH 7.4) at a concentration of 25 mg/mL, and the polymers were eluted with Tris buffer (20 mM, pH 7.4) at a rate of 0.5 mL/min. Molecular weights were calibrated with standard poly[N-(2-hydroxypropyl)methacrylamide] (pHPMA).

TABLE 1

| | | | | Buffering |
|---|---|---|---|---|
| | Mₙ | M_{w} | PDI | Capacity |
| Polymer | (kDa) | (kDa) | (M_{w}/Mₙ) | (%) |
| poly(CBA-DAE) | 2.85 | 3.34 | 1.17 | 52.61 |
| poly(CBA-DAB) | 4.23 | 4.72 | 1.12 | 61.20 |
| poly(CBA-DAH) | 3.12 | 3.52 | 1.13 | 55.65 |

Results (Table 1) showed that the range of the weight average molecular weight (M_{w}) of these polymers was from 3.34˜4.72 kDa, while the range of the number average molecular weight (Mₙ) was from 2.85˜4.23 kDa. The low polydispersity index (PDI=M_{w}/Mₙ), ranging from 1.12˜1.17, indicated that these polydisulfide amines have a narrow molecular weight distribution.

The buffering capacities of the poly(disulfide amine)s were determined by acid-base titration (FIG. 2). Briefly, 10 mL polymer solution was adjusted initially to pH 11.0 by 0.1 M NaOH. Then the basic polymer solutions were titrated to pH 3.0 with aliquots of 0.01 M HCl. The pH of the solutions was measured after each addition. The buffering capacity is defined as the percentage of amine groups becoming protonated from pH 7.4 to 5.1 and can be calculated from the following equation, C. Lin et al., supra:

Buffering capacity(%)=[(ΔV_{HCl}×0.01 M)/(Nmol)]×100.

Here ΔV_{HCl} is the volume of 0.01 M HCl solution that brought the pH value of the polymer solution from 7.4 to 5.1, and Nmol is the total moles of amine groups in the known amount of poly(disulfide amine)s.

### Example 6

Polyplexes were prepared by vortexing 1 μg pDNA (25 μL, 40 μg/mL) solution with an equal volume of polymer solution at predetermined nitrogen/phosphate (N/P) ratios, followed by a 30 min incubation. The polyplexes were then diluted in 2 mL of dust-free diH₂O, and the average particle sizes of polyplexes were measured using a BI-200SM Dynamic Light Scattering (DLS, Brookhaven Instrument Corporation, Holtsville, N.Y.) at 633 nm incident beam. Measurements were made at 25° C. at an angle of 90°. Measurements for each sample were repeated three times and reported as mean values±standard deviations (FIG. 3).

### Example 7

Agarose gel (1%, w/v) containing 0.5 μg/mL ethidium bromide (EtBr) was prepared in TAE (Tris-Acetate-EDTA) buffer. Poly(disulfide amine)s/DNA complexes at predetermined N/P ratios were prepared in HEPES buffer as described in Example 6. The samples were mixed with 6× loading dye and the mixtures were loaded onto an agarose gel. The gel was run at 100 V for 30 min and the location of DNA bands was visualized with a UV illuminator using a Gel Documentation System (Bio-Rad, Hercules, Calif.). The DNA release from poly(disulfide amine)s/DNA polyplexes was evaluated by incubating polyplexes with 5 mM DTT at 37° C. for 1 hr. The samples were then analyzed by gel electrophoresis as described above (FIGS. 4A-4F).

### Example 8

Synthetic poly(disulfide amine)-mediated transfection was evaluated on 293T cells (human renal epithelial cell line, ATCC), Hela cells (human cervical cancer cell line, ATCC), NIH3T3 (mouse embryonic fibroblasts, ATCC) and C2C12 cells (mouse myoblast cell line, ATCC) using the plasmid, pCMV-Luc, as a reporter. Cells were maintained in DMEM containing 10% FBS, streptomycin (100 μg/mL) and penicillin (100 units/mL) at 37° C. in a humidified atmosphere with 5% CO₂. Cells were seeded 24 hrs prior to transfection in 24-well plates at initial densities of 8.0×10⁴, 4.0×10⁴, 4.0×10⁴, and 3.5×10⁴ cells/well for 293T, Hela, NIH3T3 and C2C12, respectively. DNA was complexed with the poly(CBA-DAE), poly(CBA-DAB), poly(CBA-DAH), and bPEI polymers at predetermined N/P ratios in HEPES buffer and incubated for 30 min before use. At the time of transfection, the medium in each well was replaced with fresh serum-free medium. Polyplexes (0.5 μg DNA/well) were incubated with the cells for 4 hrs at 37° C. The medium was then replaced with 500 μL of fresh complete medium and cells were incubated for additional 44 hrs. The cells were then washed with pre-warmed PBS, treated with 200 μL cell lysis buffer and subjected to a freezing-thawing cycle. Cellular debris was removed by centrifugation at 14,000 g for 5 min. The luciferase activity in cell lysates (25 μL) was measured using a luciferase assay kit (100 μL luciferase assay buffer) on a luminometer (Dynex Technologies Inc., Chantilly, Va.). The relative luminescence unit (RLU) of luciferase expression was normalized against protein concentration in the cell extracts, measured by a BCA protein assay kit (Pierce, Rockford, Ill.). All transfection assays were carried out in triplicate (FIGS. 5A-5D).

### Example 9

NIH3T3 cells were seeded in a 24-well plate at a density of 4.0×10⁴ cells/well and incubated for 24 hrs. DNA was complexed with the poly(CBA-DAE), poly(CBA-DAB), poly(CBA-DAH), and bPEI at predetermined N/P ratios in HEPES buffer and incubated for 30 min before use. Polyplexes (0.5 μg DNA/well) were incubated with the cells for 4 hrs in serum-free medium followed by 20 hrs in complete medium. MTT solution (50 μL, 2 mg/mL) was then added and cells were further incubated for 2 hrs. The medium was removed and 300 μL DMSO was then added to each well. The absorption was measured at 570 nm using a microplate reader (Model 680, Bio-Rad Lab, Hercules, Calif.). The percentage relative cell viability was determined relative to control (untreated) cells, which were not exposed to the transfection system and taken as 100% cell viability. All cytotoxicity experiments were performed in triplicate (FIG. 6).

The subject matter claimed is:

1. A composition represented by the formula 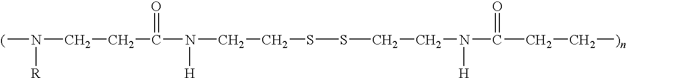
wherein n is about 1 to about 100 and R is (CH₂)ₘNH₂, wherein m is about 1 to about 18.

2. The composition of claim 1 wherein R is (CH₂)₂NH₂.

3. The composition of claim 1 wherein R is (CH₂)₄NH₂.

4. The composition of claim 1 wherein R is (CH₂)₆NH₂.

5. A complex comprising a selected nucleic acid bonded to a composition represented by the formula 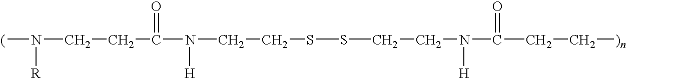
wherein n is about 1 to about 100 and R is (CH₂)ₘNH₂, wherein m is about 1 to about 18.

6. The complex of claim 5 wherein R is (CH₂)₂NH₂.

7. The complex of claim 5 wherein R is (CH₂)₄NH₂.

8. The complex of claim 5 wherein R is (CH₂)₆NH₂.

9. The complex of claim 5 wherein the selected nucleic acid comprises a plasmid.

10. The complex of claim 5 wherein the selected nucleic acid comprises siRNA.

11. The complex of claim 5 wherein the selected nucleic acid comprises an oligonucleotide.

12. A method for transfecting mammalian cells, the method comprising contacting selected mammalian cells with a complex comprising a nucleic acid bonded to a composition represented by the formula 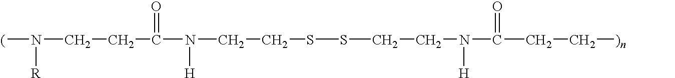
wherein n is about 1 to about 100 and R is (CH₂)ₘNH₂, wherein m is about 1 to about 18.

13. The method of claim 12 wherein R is (CH₂)₂NH₂.

14. The method of claim 12 wherein R is (CH₂)₄NH₂.

15. The method of claim 12 wherein R is (CH₂)₆NH₂.
